(19) United States
(12) Patent Application Publication
(10) Pub. No.: US 2023/0042612 A1
(43) Pub. Date: Feb. 9, 2023

(54) METHODS OF CONTROLLING OR PREVENTING INFESTATION OF PLANTS BY A PHYTOPATHOGENIC MICROORGANISM OF THE GENUS MACROPHOMINA SPP.

(71) Applicant: SYNGENTA CROP PROTECTION AG, Basel (CH)

(72) Inventor: Jon HAMILL, Greensboro, NC (US)

(73) Assignee: SYNGENTA CROP PROTECTION AG, Basel (CH)

(21) Appl. No.: 17/783,455

(22) PCT Filed: Dec. 3, 2020
(86) PCT No.: PCT/EP2020/084513

§ 371 Date: Jun. 8, 2022

(30) Foreign Application Priority Data
Dec. 10, 2019 (EP) 19214692.6

(51) Int. Cl. A01N 43/40; A01P 3/00; C07D 213/82; C07D 213/81

(52) U.S. Cl. CPC A01N 43/40; A01P 3/00; C07D 213/82; C07D 213/81; A01G 22/25

(57) ABSTRACT

The present invention relates to methods for controlling or preventing infestation of a plant by a phytopathogenic microorganism of the genus Macrophomina spp., comprising applying to a crop of plants, the locus thereof, or propagation material thereof, a compound according to formula (I), wherein R1, R2, R3, R4, R5, Y, A, B are as defined herein.

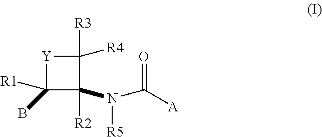

### TECHNICAL FIELD

The present invention relates to methods for controlling or preventing infestation of a plant by a phytopathogenic microorganism of the genus Macrophomina spp.

### BACKGROUND

Macrophomina spp. is a fungus that infects nearly 500 plant species in more than 100 families. The pathogen affects the fibrovascular system of the roots and basal internodes of its host, blocking the transport of water and nutrients to the upper parts of the plants. As a result, progressive wilting, premature dying, loss of vigor, and reduced yield are characteristic symptoms of the infection. The fungus also causes many diseases like damping off, seedling blight, collar rot, stem rot, charcoal rot, basal stem rot, and root rot.

The current invention provides further improved methods for controlling or preventing infestation of plants by a phytopathogenic microorganism of the genus Macrophomina spp.

### DESCRIPTION OF THE EMBODIMENTS

Cyclobutylcarboxamide compounds and processes for their preparation have been disclosed in WO2013/143811 and WO2015/003951. It has now been surprisingly found that particular cyclobutylcarboxamide compounds disclosed in WO2013/143811 and/or WO2015/003951 are highly effective at controlling or preventing the infestation of plants by a phytopathogenic microorganism of the genus Macrophomina spp. These highly effective compounds thus represent an important new solution for farmers to control or prevent infestation of plants by a phytopathogenic microorganism of the genus Macrophomina spp.

Hence, as embodiment 1, there is provided a method of controlling or preventing infestation of plants by a phytopathogenic microorganism of the genus Macrophomina spp. comprising applying to a crop of plants, the locus thereof, or propagation material thereof, a compound according to formula (I)

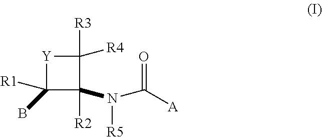

wherein

Y is O, C═O, or CR12R13;

A is a 5- or 6-membered heteroaromatic ring containing 1 to 3 heteroatoms, each independently selected from oxygen, nitrogen and sulphur, or a phenyl ring; the heteroaromatic ring or the phenyl being optionally substituted by one or more R6;

R6 is, independently of each other, halogen, cyano, C1-C4-alkyl, C1-C4-haloalkyl, C1-C4-alkoxy, C1-C4-haloalkoxy, C1-C4-haloalkylthio, C1-C4-alkoxy-C1-4-alkyl or C1-C4-haloalkoxy-C1-C4-alkyl; R1, R2, R3, R4, R12 and R13, independently of each other, are hydrogen, halogen, cyano, C1-C4-alkyl, C1-C4-alkoxy or C1-C4-haloalkyl,

R5 is hydrogen, methoxy or hydroxyl,

B is phenyl substituted by one or more R8,

R8 is, independently of each other, halogen, cyano or a group -L-R9, where each L is independently of each other a bond, —O—, —OC(O)—, —NR7-, —NR7CO—, —NR7S(O)n-, —S(O)n-, —S(O)nNR7-, —COO— or CONR7-,

n is 0, 1 or 2,

R7 is hydrogen, C1-C4-alkyl, C1-C4-haloalkyl, benzyl or phenyl, where benzyl and phenyl is unsubstituted or substituted with halogen, cyano, C1-C4-alkyl or C1-C4-haloalkyl,

R9 is, independently of each other, C1-C6-alkyl, which is unsubstituted or substituted by one or more R10, C3-C6-cycloalkyl, which is unsubstituted or substituted by one or more R10, C6-C14-bicycloalkyl, which is unsubstituted or substituted by one or more R10, C2-C6-alkenyl, which is unsubstituted or substituted by one or more R10, C2-C6-alkynyl, which is unsubstituted or substituted by one or more R10, phenyl, which is unsubstituted or substituted by R10, or heteroaryl, which is unsubstituted or substituted by one or more R10,

R10 is, independently of each other, halogen, cyano, C1-C4-alkyl, C1-C4-haloalkyl, C1-C4-alkoxy, C1-C4-haloalkoxy, C1-C4-alkylthio, C1-C4-haloalkylthio, C3-C6-alkenyloxy, or C3-C6-alkynyloxy; or a salt or N-oxide thereof;

wherein B and A-CO—NR5 are cis to each other on the four-membered ring,

or a tautomer or stereoisomer of these compounds.

More preferred methods according to embodiment 1 are given in the embodiments below.

As embodiment 2, there is provided a method according to embodiment 1 wherein Y is O or CH2;

A is a 6-membered heteroaromatic ring containing 1 to 2 nitrogen atoms, or a phenyl ring; the heteroaromatic ring or the phenyl being optionally substituted by one or more R6;

R6 is, independently of each other, halogen, cyano, C1-C4-alkyl, C1-C4-haloalkyl, or C1-C4-haloalkoxy;

R1, R2, R3, R4, and R5 are each hydrogen;

B is phenyl substituted by one or more R8;

R8 is, independently of each other, selected from halogen, cyano, C1-C4-alkyl, C1-C4-haloalkyl, C1-C4-haloalkoxy and C3-C6-cycloalkyl.

As embodiment 3, there is provided a method according to either embodiment 1 or embodiment 2 wherein A is a 6-membered heteroaromatic ring containing 1 to 2 nitrogen atoms and having 1 to 3 substituents selected from R6, or a phenyl ring having 1 or 3 substitutents selected from R6.

As embodiment 4, there is provided a method according to any one of embodiments 1 to 3 wherein B is a phenyl substituted by 1 to 3 substitutents R8.

As embodiment 5, there is provided a method according to any one of embodiments 1 to 4 wherein B is a phenyl substituted by 1 to 3 substituents, independently selected from fluoro, chloro, trifluoromethyl, cyclopropyl, difluoromethoxy and trifluoromethoxy;

A is a phenyl, pyridyl or pyrazinyl, which rings, independently of each other, are unsubstituted or substituted by 1 to 3 substituents, independently selected from chloro, bromo, fluoro, methyl, cyano, and trifluoromethyl, Y is O or CH2, and R1, R2, R3, R4 and R5 are each hydrogen.

As embodiment 6, there is provided a method according to any one of embodiments 1 to 5 wherein Y is CH2;

B is a mono or di-halogen substituted phenyl;

A is selected from phenyl, pyrazinyl and pyridyl, each of which is mono or di-substituted by substituents independently selected from halogen and C1-C4-haloalkyl;

R1, R2, R3, R4 and R5 are each hydrogen.

Compounds of formula (I) as disclosed in any one of embodiments 1 to 6 represent the cis racemate: the phenyl ring on the left hand side and the A-C(═O)—NH group on the right hand side are cis to each other on the cyclobutyl ring:

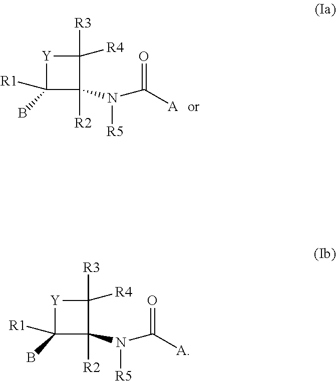

Thus, the racemic compound of formula (I) is a 1:1 mixture of the compounds of formula (Ia) and (Ib). The wedged bonds shown in the compounds of formula (Ia) and (Ib) represent absolute stereochemistry, whereas the thick straight bonds such as those shown for the compounds of formula (I) represent relative stereochemistry in racemic compounds.

It has also surprisingly been found that one enantiomer of the compounds of formula (I) is particularly useful in controlling or preventing the infestation of plants by a phytopathogenic microorganism of the genus Macrophomina spp.

Thus, as embodiment 7, there is provided the method according to any one of embodiments 1 to 6 wherein the compound is of formula (Ia)

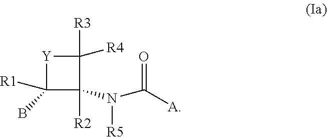

A skilled person is aware that according to the method of embodiment 2, the compound of formula (Ia) is generally applied as part of a pesticidal composition. Hence, as embodiment 8, there is provided a method of controlling or preventing infestation of plants by a phytopathogenic microorganism of the genus Macrophomina spp., comprising applying to a crop of plants, the locus thereof, or propagation material thereof a pesticidal composition comprising a compound according to anyone of embodiments 1-7 and one or more formulation adjuvants. As embodiment 9, there is provided a method of controlling or preventing infestation of plants by a phytopathogenic microorganism of the genus Macrophomina spp., comprising applying to a crop of plants, the locus thereof, or propagation material thereof a pesticidal composition comprising a compound of formula (Ia) and one or more formulation adjuvants. In a method according to embodiment 9, for pesticidal compositions comprising both a compound of formula (Ia) and a compound of formula (Ib), the ratio of the compound of formula (Ia) to its enantiomer (the compound of formula (Ib)) must be greater than 1:1. Preferably, the ratio of the compound of formula (Ia) to the compound of formula (Ib) is greater than 1.5:1, more preferably greater than 2.5:1, especially greater than 4:1, advantageously greater than 9:1, desirably greater than 20:1, in particular greater than 35:1.

Mixtures containing up to 50%, preferably up to 40%, more preferably up to 30%, especially up to 20%, advantageously up to 10%, desirably up to 5%, in particular up to 3%, of the trans stereoisomers of the compounds of formula (I) (i.e. wherein the B and the A-C(═O)—NH groups are trans to each other) are also understood to be part of this invention. Preferably, the ratio of the compound of formula (I) to its trans isomer is greater than 1.5:1, more preferably greater than 2.5:1, especially greater than 4:1, advantageously greater than 9:1, desirably greater than 20:1, in particular greater than 35:1.

Preferably, in a composition comprising the compound of formula (Ia), its trans isomer (i.e. wherein the B and the A-CO—NR2 groups are trans to each other) and the compound of formula (Ib), the composition comprises the compound of formula (Ia) in a concentration of at least 50%, more preferably 70%, even more preferably 85%, in particular over 90%, and particularly preferably over 95%, each based on the total amount of compound of formula (Ia), its trans isomer and the compound of formula (Ib).

Further, as embodiment 10, there is provided a method of controlling or preventing infestation of plants by a phytopathogenic microorganism of the genus Macrophomina spp., comprising applying to a crop of plants, the locus thereof, or propagation material thereof, a compound according to formula (Ic)

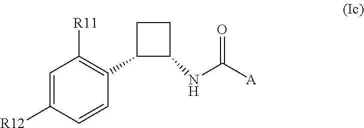

wherein

R11 and R12 are independently selected from halogen;

A is pyridyl which is substituted by one or two substituents independently selected from halogen and C₁-C₄-haloalkyl.

As embodiment 11, there is provided a method according to embodiment 10, wherein

R11 and R12 are independently selected from chloro and fluoro;

A is pyrid-2-yl or pyrid-3-yl, which is substituted by one or two C₁-C₄-haloalkyl substituents.

As embodiment 12, there is provided a method according to embodiments 10 or 11, wherein A is selected from

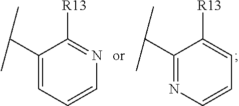

R13 is C₁-C₄-haloalkyl, preferably trifluoromethyl.

As embodiment 13, there is provided a method according to any one of embodiments 10 to 12 wherein the compound is selected from any one of compounds 1 to 12 of formula (Ic)

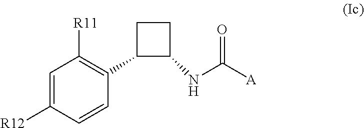

wherein R11, R12 and A are as defined in the following table:

| Compound | A | R11 | R12 |
|---|---|---|---|
| 1 | 2-trifluoromethyl-pyrid-3-yl | Cl | Cl |
| 2 | 3-trifluoromethyl-pyrid-2-yl | Cl | Cl |
| 3 | 3-trifluoromethyl-pyrid-2-yl | F | F |
| 4 | 3-trifluoromethyl-pyrid-2-yl | Cl | F |
| 5 | 3-chloro-pyrid-2-yl | Cl | Cl |
| 6 | 2-methyl-pyrid-3-yl | Cl | Cl |
| 7 | 2-trifluoromethyl-pyrid-3-yl | Cl | F |

As embodiment 14, there is provided the method according to any one of embodiments 1 to 13 comprising the steps

providing a composition comprising a compound as defined in any one of embodiments 1 to 13; applying the composition to a propagation material;

planting the propagation material.

As embodiment 15, there is provided the method according to any one of embodiments 1 to 13 comprising the steps

providing a composition comprising a compound as defined in any one of embodiments 1 to 13; applying the composition to a crop of plants or the locus thereof.

As embodiment 16, there is provided the use of a compound as defined in any one of embodiments 1 to 13 for controlling or preventing infestation of plants by a phytopathogenic microorganism of the genus Macrophomina spp.

As embodiment 17, there is provided the use of a compound as defined in any one of embodiments 1 to 13 for controlling or preventing infestation of plants by a phytopathogenic microorganism of the genus macrophomina spp., particularly wherein the phytopathogenic microorganism is Macrophomina phaseolina or Macrophomina limbalis, more particularly is Macrophomina phaseolina.

As embodiment 18, there is provided a method for growing strawberry plants comprising applying or treating strawberry or a propagation material thereof with a compound as defined in any one of claims 1 to 13.

As embodiment 19, there is provided a method or use according to any one of embodiments 1 to 17, wherein the plant is selected from

Abelmoschus

Abies

Abutilon

Acer

Allium

Amaranthus

Ambosia

Antirrhinum

Apocynum

Arachis

Arrhenatherum

Asclepias

Asparagus

Avena

Begonia

Beta

Bidens

Bouteloua

Brassica

Campanula

Canjanus

Cannabis

Capsicum

Cassia

Catalpa

Celosia

Chamaecyparis

Chenopodium

Chrysanthemum

Cicer

Cirsium

Citrullus

Citrus

Conyza

Cornus

Crotalaria

Cucumis

Cucurbita

Cupressus

Cyamopsis

Dahlia

Datura

Dichondra

Elymus

Erigeron

Eryngium

Eupatorium

Euphorbia

Fagopyrum

Strawberry

Glycine

Gossypium

Hedera

Helianthus

Hibiscus

Ipomoea

Juniperus

Koelreuteria

Kummerowia

Lactuca

Lespedeza

Ligustrum

Lilium

Lotus

Lupinus

Lycopersicon

Malva

Medicago

Melilotus

Muhlenbergia

Nicandra

Nicotiana

Nyssa

Oenothera

Opuntia

Parthenium

Phaseolus

Phlox

Picea

Pinus

Pisum

Polygonum

Prunus

Pseudotsuga

Pueraria

Pyracantha

Quercus

Rhododendron

Ricinus

Robinia

Roystonea

Rudbeckia

Salvia

Santolina

Schefflera

Senna

Sequoiadendron

Sesamum

Sesbania

Setaria

Sida

Solanum

Solidago

Sorghum

Strophostyles

Sugarbeet

Tagetes

Thuja

Trifolium

Tristania

Verbena

Vicia

Vigna

Vitis

Zea and

Zinnia.

As embodiment 20, there is provided a method or use according to any one of embodiments 1 to 18, wherein the plant is selected from peanut, cabbage, pepper, chickpea, soybean, sunflower, sweet potato, sugarbeet, alfalfa, sesame, potato, sorghum, wheat, corn and strawberry.

As embodiment 21, there is provided a method according to any one of claims 1 to 20 wherein the plant is strawberry and wherein the phytopathogenic microorganism is Macrophomina phaseolina.

The preparation of the compounds as defined in the methods of any one of embodiments 1 to 13 has been disclosed in WO2013/143811 and WO2015/003951 which are incorporated herein by reference.

### Definitions

The term “halogen” represents fluoro, chloro, bromo or iodo, particularly fluoro, chloro or bromo.

The term “alkyl” or “alk” as used herein either alone or as part of a larger group (such as alkoxy, alkylthio, alkoxycarbonyl and alkylcarbonyl) is a straight or branched chain and is, for example, methyl, ethyl, n-propyl, n-butyl, isopropyl, sec-butyl, isobutyl, tert-butyl, pentyl, iso-pentyl or n-hexyl. The alkyl groups are suitably C₁-C₄-alkyl groups.

“Haloalkyl” as used herein are alkyl groups as defined above which are substituted with one or more of the same or different halogen atoms and are, for example, CF₃, CF₂Cl, CF₂H, CCl₂H, FCH₂, ClCH₂, BrCH₂, CH₃CHF, (CH₃)₂CF, CF₃CH₂ or CHF₂CH₂.

The methods and uses according to any one of embodiments 1 to 18 are preferably for controlling or preventing infestation of the crop by the phytopathogenic microorganism Cercospora that are resistant to other fungicides. Cercospora that are “resistant” to a particular fungicide refer e.g. to strains of Cercospora fungi that are less sensitive to that fungicide compared to the expected sensitivity of the same species of Cercospora fungi. The expected sensitivity can be measured using e.g. a strain that has not previously been exposed to the fungicide.

Application according to the methods or uses according to any one of embodiments 1 to 18 is preferably to a crop of plants, the locus thereof or propagation material thereof. Preferably application is to a crop of plants or propagation material thereof, more preferably to propagation material.

Application of the compounds of the invention can be performed according to any of the usual modes of application, e.g. foliar, drench, soil, in furrow etc.

The compounds as defined in any one of embodiments 1 to 13 are preferably used for pest control at 1 to 500 g/ha, preferably 10-70 g/ha.

The compounds as defined in any one of embodiments 1 to 13 are suitable for use on any peanut plant, including those that have been genetically modified to be resistant to active ingredients such as herbicides, or to produce biologically active compounds that control infestation by plant pests.

Generally, a compound as defined in any one of embodiments 1 to 13 is used in the form of a composition (e.g. formulation) containing a carrier. A compound as defined in any one of embodiments 1 to 13 and compositions thereof can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ulv) liquid, ultra low volume (ulv) suspension, water dispersible granules or tablets, water dispersible powder for slurry treatment, water soluble granules or tablets, water soluble powder for seed treatment and wettable powder.

A formulation typically comprises a liquid or solid carrier and optionally one or more customary formulation auxiliaries, which may be solid or liquid auxiliaries, for example unepoxidized or epoxidized vegetable oils (for example epoxidized coconut oil, rapeseed oil or soya oil), antifoams, for example silicone oil, preservatives, clays, inorganic compounds, viscosity regulators, surfactant, binders and/or tackifiers. The composition may also further comprise a fertilizer, a micronutrient donor or other preparations which influence the growth of plants as well as comprising a combination containing the compound of the invention with one or more other biologically active agents, such as bactericides, fungicides, nematicides, plant activators, acaricides, and insecticides.

The compositions are prepared in a manner known per se, in the absence of auxiliaries for example by grinding, screening and/or compressing a solid compound of the present invention and in the presence of at least one auxiliary for example by intimately mixing and/or grinding the compound of the present invention with the auxiliary (auxiliaries). In the case of solid compounds of the invention, the grinding/milling of the compounds is to ensure specific particle size.

Examples of compositions for use in agriculture are emulsifiable concentrates, suspension concentrates, microemulsions, oil dispersibles, directly sprayable or dilutable solutions, spreadable pastes, dilute emulsions, soluble powders, dispersible powders, wettable powders, dusts, granules or encapsulations in polymeric substances, which comprise—at least—a compound as defined in any one embodiments 1 to 13 and the type of composition is to be selected to suit the intended aims and the prevailing circumstances.

As a rule, the compositions comprise 0.1 to 99%, especially 0.1 to 95%, of compound as defined in any one of embodiments 1 to 13 and 1 to 99.9%, especially 5 to 99.9%, of at least one solid or liquid carrier, it being possible as a rule for 0 to 25%, especially 0.1 to 20%, of the composition to be surfactants (% in each case meaning percent by weight). Whereas concentrated compositions tend to be preferred for commercial goods, the end consumer as a rule uses dilute compositions which have substantially lower concentrations of active ingredient.

Examples of foliar formulation types for pre-mix compositions are:

GR: Granules

WP: wettable powders

WG: water dispersable granules (powders)

SG: water soluble granules

SL: soluble concentrates

EC: emulsifiable concentrate

EW: emulsions, oil in water

ME: micro-emulsion

SC: aqueous suspension concentrate

CS: aqueous capsule suspension

OD: oil-based suspension concentrate, and

SE: aqueous suspo-emulsion.

Whereas, examples of seed treatment formulation types for pre-mix compositions are:

WS: wettable powders for seed treatment slurry

LS: solution for seed treatment

ES: emulsions for seed treatment

FS: suspension concentrate for seed treatment

WG: water dispersible granules, and

CS: aqueous capsule suspension.

Examples of formulation types suitable for tank-mix compositions are solutions, dilute emulsions, suspensions, or a mixture thereof, and dusts.

As with the nature of the formulations, the methods of application, such as foliar, drench, spraying, atomizing, dusting, scattering, coating or pouring, are chosen in accordance with the intended objectives and the prevailing circumstances.

The tank-mix compositions are generally prepared by diluting with a solvent (for example, water) the one or more pre-mix compositions containing different pesticides, and optionally further auxiliaries.

Suitable carriers and adjuvants can be solid or liquid and are the substances ordinarily employed in formulation technology, e.g. natural or regenerated mineral substances, solvents, dispersants, wetting agents, tackifiers, thickeners, binders or fertilizers.

Generally, a tank-mix formulation for foliar or soil application comprises 0.1 to 20%, especially 0.1 to 15%, of the desired ingredients, and 99.9 to 80%, especially 99.9 to 85%, of a solid or liquid auxiliaries (including, for example, a solvent such as water), where the auxiliaries can be a surfactant in an amount of 0 to 20%, especially 0.1 to 15%, based on the tank-mix formulation.

Typically, a pre-mix formulation for foliar application comprises 0.1 to 99.9%, especially 1 to 95%, of the desired ingredients, and 99.9 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Normally, a tank-mix formulation for seed treatment application comprises 0.25 to 80%, especially 1 to 75%, of the desired ingredients, and 99.75 to 20%, especially 99 to 25%, of a solid or liquid auxiliaries (including, for example, a solvent such as water), where the auxiliaries can be a surfactant in an amount of 0 to 40%, especially 0.5 to 30%, based on the tank-mix formulation.

Typically, a pre-mix formulation for seed treatment application comprises 0.5 to 99.9%, especially 1 to 95%, of the desired ingredients, and 99.5 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Whereas commercial products will preferably be formulated as concentrates (e.g., pre-mix composition (formulation)), the end user will normally employ dilute formulations (e.g., tank mix composition).

Preferred seed treatment pre-mix formulations are aqueous suspension concentrates. The formulation can be applied to the seeds using conventional treating techniques and machines, such as fluidized bed techniques, the roller mill method, rotostatic seed treaters, and drum coaters. Other methods, such as spouted beds may also be useful. The seeds may be presized before coating. After coating, the seeds are typically dried and then transferred to a sizing machine for sizing. Such procedures are known in the art. The compounds of the present invention are particularly suited for use in soil and seed treatment applications.

In general, the pre-mix compositions of the invention contain 0.5 to 99.9 especially 1 to 95, advantageously 1 to 50, % by mass of the desired ingredients, and 99.5 to 0.1, especially 99 to 5, % by mass of a solid or liquid adjuvant (including, for example, a solvent such as water), where the auxiliaries (or adjuvant) can be a surfactant in an amount of 0 to 50, especially 0.5 to 40, % by mass based on the mass of the pre-mix formulation.

Furthermore, there is provided a method of controlling or preventing infestation of strawberry plants by phytopathogenic microorganisms selected from BOTRYTIS CINEREA and PODOSPHAERA MACULARIS, comprising applying to a crop of plants, the locus thereof, or propagation material thereof, a compound according to any one of embodiments 1 to 13.

The invention will now be illustrated by the following non-limiting Examples. All citations are incorporated by reference.

### Biological Examples

### Effect of Different Fungicide Treatments Against Macrophomina sp.

A strawberry pot trial was carried out in the greenhouse Vero Beach, United States to evaluate the efficacy of different compounds against Macrophomina phaseolina.

The plants were planted in 4 L pots with Vero Beach Mix (50% potting media and 50% pasteurized sand). The plants were bare root strawberry of the variety ‘Sweet Ann’. The strawberry plants were partially planted, then were inoculated with 2 grams of the infested millet. The inoculum was grown on twice sterilized millet for 1-2 weeks. The isolate used was originally isolated from strawberry. After the inoculation the roots were completely covered. At the day of planting each plant was drench applied with 100 mL of compound solution. The application dose was calculated based on a plant spacing of 30.5 cm by 45.7 cm. The plants were grown in a greenhouse that averaged 32° C. in the day and 21° C. at night. Plants were watered daily. The disease severity was evaluated after 44 and 65 days after application using a IS50-5 index scale (5=severe damage, 0=no damage).

### Trial Location:

| Trial Location | Sown | Variety | Resistance Status |
|---|---|---|---|
| Vero Beach, FL, United | ¹ Aug. 2018 | Sweet Ann | Susceptible |
| States | | | |

### Treatment List—Field Trials:

| | | Active | |
|---|---|---|---|
| | | ingredient | |
| | | (Al) Rate | Application |
| Treatment | | (g Al/ha) | method |
| 1 | CHECK | — | — |
| 2 | COMPOUND 1 SC450 | 250 g Al/ha | DRENCH; AT |
| | | | PLANTING; SOIL |
| 3 | Commercial standard: Succinate | 250 g Al/ha | DRENCH; AT |
| | dehydrogenase inhibitor SC500 | | PLANTING; SOIL |
| | (Fluopyram) | | |

### Crops and Targets Occurred in the Trial:

| | Latin name | Common name |
|---|---|---|
| Target | MACROPHOMINA PHASEOLI | STRAWBERRY |
| Crop | FRAGARIA SP. | |

### Crop Description:

| | |
|---|---|
| Test Crop | STRAWBERRY |
| Variety | Sweet Ann |
| Sowing or Planting Date | Jan. 8, 2018 |

### Trial Layout:

| Trial Environment (Test | |
|---|---|
| Method) | Greenhouse |
| Experimental Design | RANDOMIZED COMPLETE BLOCK |
| # replications | 6 |

### Application Details:

| Application Date | Jan. 8, 2018 |
|---|---|
| Appl. Equipment Type | DRENCH—SOIL |
| Spray Volume | 100 ML/PLANT |
| Treatments applied | 2, 3 |

### Assessments:

| | Pest severity (IS 0-5; 0 = no | |
|---|---|---|
| | damage, 5 = severe damage), | |
| | significantly different | |
| | (Treatments with no letter in | % efficacy |
| | common are significantly | based on |
| | different at the 5% probability | disease |
| | level) | incidence |
| UNTREATED | 3,A | 0.00 |
| COMPOUND 1 | 0,B | 100.00 |
| Commercial standard: | 1.67,A | 44.33 |
| Succinate | | |
| dehydrogenase | | |
| inhibitor SC500 | | |
| (Fluopyram) | | |

Pest Severity, 44 Days after Planting

| | Pest severity (IS 0-5; 0 = no | |
|---|---|---|
| | damage, 5 = severe damage), | |
| | significantly different | |
| | (Treatments with no letter in | % efficacy |
| | common are significantly | based on |
| | different at the 5% probability | disease |
| | level) | incidence |
| UNTREATED | 5,A | 0.00 |
| COMPOUND 1 | 0.83,B | 83.40 |
| Commercial standard: | 4.92,A | 1.60 |
| Succinate | | |
| dehydrogenase | | |
| inhibitor SC500 | | |
| (Fluopyram) | | |

### CONCLUSION

In this trial, compound 1 showed excellent activity against Macrophomina phaseolina in strawberry over a duration of 65 days after application. Compound 2 showed moderate activity (44%) until 44 days, afterwards it lost its activity almost completely.

1. A method of controlling or preventing infestation of a plant by a phytopathogenic microorganism of the genus Macrophomina spp., comprising applying to a crop of plants, the locus thereof, or propagation material thereof, a compound according to formula (I) 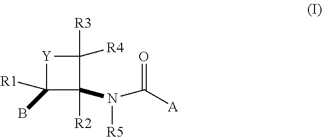
wherein
Y is O, C═O, or CR12R13;
A is a 5- or 6-membered heteroaromatic ring containing 1 to 3 heteroatoms, each independently selected from oxygen, nitrogen and sulphur, or a phenyl ring; the heteroaromatic ring or the phenyl being optionally substituted by one or more R6;
R6 is, independently of each other, halogen, cyano, C1-C4-alkyl, C1-C4-haloalkyl, C1-C4-alkoxy, C1-C4-haloalkoxy, C1-C4-haloalkylthio, C1-C4-alkoxy-C1-4-alkyl or C1-C4-haloalkoxy-C1-C4-alkyl;
R1, R2, R3, R4, R12 and R13, independently of each other, are hydrogen, halogen, cyano, C1-C4-alkyl, C1-C4-alkoxy or C1-C4-haloalkyl,
R5 is hydrogen, methoxy or hydroxyl,
B is phenyl substituted by one or more R8,
R8 is, independently of each other, halogen, cyano or a group -L-R9, where each L is independently of each other a bond, —O—, —OC(O)—, —NR7-, —NR7CO—, —NR7S(O)n-, —S(O)n-, —S(O)nNR7-, —COO— or CONR7-,
n is 0, 1 or 2,
R7 is hydrogen, C1-C4-alkyl, C1-C4-haloalkyl, benzyl or phenyl, where benzyl and phenyl is unsubstituted or substituted with halogen, cyano, C1-C4-alkyl or C1-C4-haloalkyl,
R9 is, independently of each other, C1-C6-alkyl, which is unsubstituted or substituted by one or more R10, C3-C6-cycloalkyl, which is unsubstituted or substituted by one or more R10, C6-C14-bicycloalkyl, which is unsubstituted or substituted by one or more R10, C2-C6-alkenyl, which is unsubstituted or substituted by one or more R10, C2-C6-alkynyl, which is unsubstituted or substituted by one or more R10, phenyl, which is unsubstituted or substituted by R10, or heteroaryl, which is unsubstituted or substituted by one or more R10,
R10 is, independently of each other, halogen, cyano, C1-C4-alkyl, C1-C4-haloalkyl, C1-C4-alkoxy, C1-C4-haloalkoxy, C1-C4-alkylthio, C1-C4-haloalkylthio, C3-C6-alkenyloxy, or C3-C6-alkynyloxy;
or a salt or N-oxide thereof;
wherein B and A-CO—NR5 are cis to each other on the four-membered ring,
or a tautomer or stereoisomer of these compounds.

2. The method according to claim 1 wherein Y is O or CH2;
A is a 6-membered heteroaromatic ring containing 1 to 2 nitrogen atoms, or a phenyl ring; the heteroaromatic ring or the phenyl being optionally substituted by one or more R6;
R6 is, independently of each other, halogen, cyano, C1-C4-alkyl, C1-C4-haloalkyl, or C1-C4-haloalkoxy;
R1, R2, R3, R4, and R5 are each hydrogen;
B is phenyl substituted by one or more R8;
R8 is, independently of each other, selected from halogen, cyano, C1-C4-alkyl, C1-C4-haloalkyl, C1-C4-haloalkoxy and C3-C6-cycloalkyl.

3. A method according to claim 1 wherein A is a 6-membered heteroaromatic ring containing 1 to 2 nitrogen atoms and having 1 to 3 substituents selected from R6, or a phenyl ring having 1 or 3 substitutents selected from R6.

4. The method according to claim 1 wherein wherein B is a phenyl substituted by 1 to 3 substitutents R8.

5. The method according to claim 1 wherein B is a phenyl substituted by 1 to 3 substituents, independently selected from fluoro, chloro, trifluoromethyl, cyclopropyl, difluoromethoxy and trifluoromethoxy;
A is a phenyl, pyridyl or pyrazinyl, which rings, independently of each other, are unsubstituted or substituted by 1 to 3 substituents, independently selected, from chloro, bromo, fluoro, methyl, cyano, and trifluoromethyl, Y is O or CH2, and R1, R2, R3, R4 and R5 are each hydrogen.

6. The method according to claim 1 wherein Y is CH2;
B is a mono or di-halogen substituted phenyl;
A is selected from phenyl, pyrazinyl and pyridyl, each of which is mono or di-substituted by substituents independently selected from halogen and C1-C4-haloalkyl;
R1, R2, R3, R4 and R5 are each hydrogen.

7. The method according to claim 1, wherein the compound is a compound of formula (Ic) 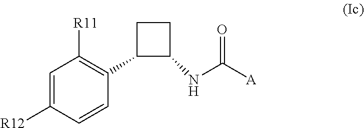
wherein
R11 and R12 are independently selected from halogen;
A is pyridyl which is substituted by one or two substituents independently selected from halogen and C₁-C₄-haloalkyl.

8. The method according to claim 7 wherein
R11 and R12 are independently selected from chloro and fluoro;
A is pyrid-2-yl or pyrid-3-yl, which is substituted by one or two C₁-C₄-haloalkyl substituents.

9. The method according to claim 1 wherein A is selected from 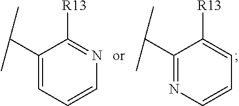
R13 is C₁-C₄-haloalkyl.

10. The method according to claim 1 wherein the compound is selected from any one of compounds 1 to 7 of formula (Ic) 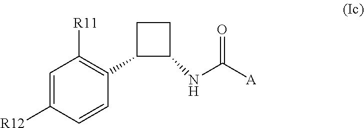
wherein R11, R12 and A are as defined in the following table:

| Compound | A | R11 | R12 |
|---|---|---|---|
| 1 | 2-trifluoromethyl-pyrid-3-yl | Cl | Cl |
| 2 | 3-trifluoromethyl-pyrid-2-yl | Cl | Cl |
| 3 | 3-trifluoromethyl-pyrid-2-yl | F | F |
| 4 | 3-trifluoromethyl-pyrid-2-yl | Cl | F |
| 5 | 3 -chloro-pyrid-2-yl | Cl | Cl |
| 6 | 2-methyl-pyrid-3-yl | Cl | Cl |
| 7 | 2-trifluoromethyl-pyrid-3-yl | Cl | F |

11. The method according to claim 1 wherein the phytopathogenic microorganism is Macrophomina phaseolina or Macrophomina limbalis.

12. The method according to claim 1 wherein the plant is selected from
Abelmoschus
Abies
Abutilon
Acer
Allium
Amaranthus
Ambosia
Antirrhinum
Apocynum
Arachis
Arrhenatherum
Asclepias
Asparagus
Avena
Begonia
Beta
Bidens
Bouteloua
Brassica
Campanula
Canjanus
Cannabis
Capsicum
Cassia
Catalpa
Celosia
Chamaecyparis
Chenopodium
Chrysanthemum
Cicer
Cirsium
Citrullus
Citrus
Conyza
Cornus
Crotalaria
Cucumis
Cucurbita
Cupressus
Cyamopsis
Dahlia
Datura
Dichondra
Elymus
Erigeron
Eryngium
Eupatorium
Euphorbia
Fagopyrum
Strawberry
Glycine
Gossypium
Hedera
Helianthus
Hibiscus
Ipomoea
Juniperus
Koelreuteria
Kummerowia
Lactuca
Lespedeza
Ligustrum
Lilium
Lotus
Lupinus
Lycopersicon
Malva
Medicago
Melilotus
Muhlenbergia
Nicandra
Nicotiana
Nyssa
Oenothera
Opuntia
Parthenium
Phaseolus
Phlox
Picea
Pinus
Pisum
Polygonum
Prunus
Pseudotsuga
Pueraria
Pyracantha
Quercus
Rhododendron
Ricinus
Robinia
Roystonea
Rudbeckia
Salvia
Santolina
Schefflera
Senna
Sequoiadendron
Sesamum
Sesbania
Setaria
Sida
Solanum
Solidago
Sorghum
Strophostyles
Sugarbeet
Tagetes
Thuja
Trifolium
Tristania
Verbena
Vicia
Vigna
Vitis
Zea and
Zinnia.

13. The method according to claim 1 wherein the plant is selected from peanut, cabbage, pepper, chickpea, soybean, sunflower, sweet potato, sugarbeet, alfalfa, sesame, potato, sorghum, wheat, corn and strawberry.

14. The method according to claim 1 wherein the plant is strawberry and wherein the phytopathogenic microorganism is Macrophomina phaseolina.

15. A method for growing strawberry plants comprising applying or treating peanut or a propagation material thereof with a compound as defined in claim 1.
